(19) United States
(12) Patent Application Publication
(10) Pub. No.: US 2003/0181520 A1
(43) Pub. Date: Sep. 25, 2003

(54) Methods of reversing and preventing cardiovascular pathologies

(76) Inventors: Mitchell Glass, (US); Jean-Claude Tardif, Laval (CA)

Correspondence Address: KING & SPALDING, 191 PEACHTREE STREET, N.E., ATLANTA, GA, 30303-1763, US

(21) Appl. No.: 10/293,399

(22) Filed: Nov. 12, 2002

Related U.S. Application Data
(60) Provisional application No. 60/347,778, filed on Nov. 9, 2001.

(51) Int. Cl. A61K 31/225

(52) U.S. Cl. 514/548

(57) ABSTRACT

The present invention is a method to increase the lumen diameter of a coronary blood vessel, in a mammal, particularly a human, that includes administering a compound of the formula

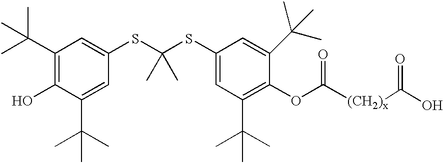

wherein x is defined as an integer between 1 and 4;

or a pharmaceutically acceptable salt, ester or prodrug thereof.

This Application claims priority to U.S. Provisional Patent Application Serial No. 60/347,778 filed on Nov. 9, 2001.

### FIELD OF THE INVENTION

The present invention describes a method to increase the lumen diameter of a blood vessel in a host mammal, particularly a human, or to treat, reverse or prevent cardiovascular diseases characterized by a decreased lumen diameter.

### BACKGROUND OF THE INVENTION

Cell migration plays an important role in wound healing, inflammation, adult respiratory distress syndrome, and malignant invasion. Migration of vascular smooth muscle cells from media to intima plays a critical role in neointima formation leading to pathological conditions including restenosis, atherosclerosis, coronary heart disease (CHD), thrombosis, myocardial infarction, stroke, smooth muscle neoplasms such as leiomyoma and leiomyosarcoma of the bowel and uterus, uterine fibroid or fibroma, and obliterative disease of vascular grafts and transplanted organs. The mechanisms of abnormal smooth muscle cell proliferation are not yet well understood.

Atherosclerosis is a cardiovascular disease in which the vessel wall is remodeled, in a process that compromises the lumen of the vessel. The atherosclerotic remodeling process involves accumulation of cells, both smooth muscle cells and monocyte/macrophage inflammatory cells, in the intima of the vessel wall. These cells take up lipid, likely from the circulation, to form a mature atherosclerotic lesion. Although the formation of these lesions is a chronic process, occurring over decades of an adult human life, the majority of the morbidity associated with atherosclerosis occurs when a lesion ruptures, releasing thrombogenic debris that rapidly occludes the artery. When such an acute event occurs in the coronary artery, myocardial infarction can ensue, and in the worst case, can result in death. Atherosclerotic coronary heart disease represents the major cause of death and cardiovascular morbidity in the western world. Despite recent declines in CHD mortality, CHD is still responsible for more than 500,000 deaths in the U.S. annually.

To date, drug intervention to treat atherosclerosis only slows the progression of the disease, and as the disease progresses invasive surgery follows. Percutaneous transluminal coronary angioplasty (PTCA) is widely used as the primary treatment modality in many patients with coronary artery disease. PTCA can relieve myocardial ischemia in patients with coronary artery disease by reducing lumen obstruction and improving coronary flow. The use of this surgical procedure is used with and without stents. Retenosis following PTCA remains a significant problem, with a significant number of patients developing restenosis within 1 to 3 months. Restenosis results in significant morbidity and mortality and frequently necessitates further interventions such as repeat angioplasty or coronary bypass surgery. No surgical intervention or post-surgical treatment (to date) has proven effective in preventing restenosis.

Compounds that reportedly suppress smooth muscle proliferation in vitro may have undesirable pharmacological side effects when used in vivo. Heparin is an example of one such compound, which reportedly inhibits smooth muscle cell proliferation in vitro but when used in vivo has the potential adverse side effect of inhibiting coagulation. Low molecular weight fragments of heparin, while having reduced anti-coagulant activity, have the undesirable pharmacological property of a short pharmacological half-life. Probucol has been shown to prevent coronary restenosis after balloon angioplasy (N Engl J Med 1997; 337:365-372), but is also known to have undesired side effect of prolonged QT interval.

U.S. Pat. No. 6,147,250 discloses therapeutic agents for the treatment of diseases, including cardiovascular diseases, which are mediated by VCAM-1. The '250 patent does not teach, mention or contemplate the reversal or prevention of CHD or pathological diseases associated with vascular smooth muscle cell proliferation or cardiovascular indications characterized by decreased lumen diameter.

U.S. Pat. No. 5,262,439 to Parthasarathy, which is assigned to AtheroGenics, Inc. discloses analogs of probucol with increased water solubility in which one or both of the hydroxyl groups are replaced with ester groups that increase the water solubility of the compound. In one embodiment, the derivative is selected from the group consisting of a mono- or di-probucol ester of succinic acid, glutaric acid, adipic acid, seberic acid, sebacic acid, azelaic acid, or maleic acid. In another embodiment, the probucol derivative is a mono- or di-ester in which the ester contains an alkyl or alkenyl group that contains a functionality selected from the group consisting of a carboxylic acid group, amine group, salt of an amine group, amide groups, amide groups, and aldehyde groups.

A series of French patents disclose that certain probucol derivatives are hypocholesterolemic and hypolipemic agents: Fr 2168137 (bis 4hydroxyphenylthioalkane esters); Fr 2140771 (tetralinyl phenoxy alkanoic esters of probucol); Fr 2140769 (benzofuryloxyalkanoic acid derivatives of probucol); Fr 2134810 (bis-(3 -alkyl-5-t-alkyl-4-thiazole-5-carboxy)phenylthio)alkanes; FR 2133024 (bis-(4-nicotinoyloxyphenylthio)propanes; and Fr 2130975 (bis(4-phenoxyalkanoyloxy)phenylthio)alkanes).

U.S. Pat. No. 5,155,250 to Parker, et al. discloses that 2,6-dialkyl-4-silylphenols are antiatherosclerotic agents. The same compounds are disclosed as serum cholesterol lowering agents in PCT Publication No. WO 95/15760, published on Jun. 15, 1995. U.S. Pat. No. 5,608,095 to Parker, et al. discloses that alkylated-4-silyl-phenols inhibit the peroxidation of LDL, lower plasma cholesterol, and inhibit the expression of VCAM-1, and thus are useful in the treatment of atherosclerosis.

A series of European patent applications to Shionogi Seiyaku Kabushiki Kaisha disclose phenol esters for use in treating arteriosclerosis. European Patent Application No. 348 203 discloses phenolic thioethers which inhibit the denaturation of LDL and the incorporation of LDL by macrophages. The compounds are useful as anti-arteriosclerosis agents. Hydroxamic acid derivatives of these compounds are disclosed in European Patent Application No. 405 788 and are useful for the treatment of arteriosclerosis, ulcer, inflammation and allergy. Carbamoyl and cyano derivatives of the phenolic thioethers are disclosed in U.S. Pat. No. 4,954,514 to Kita, et al.

U.S. Pat. No. 6,121,319, which issued on Sep. 19, 2000, and corresponding WO 98/51662 filed by AtheroGenics, Inc. and published on Nov. 18, 1998, describes certain compounds of formula having the structure

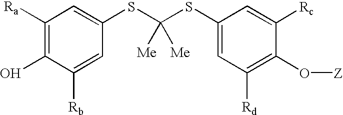

wherein:

Ra, Rb, Rc, and Rd are independently any group that does not otherwise adversely affect the desired properties of the molecule, including hydrogen, straight chained, branched, or cyclic alkyl which may be substituted, aryl, substituted aryl, heteroaryl, substituted heteroaryl, alkaryl, substituted alkaryl, aralkyl or substituted aralkyl; substituents on the Ra, Rb, Rc and Rd groups are selected from the group consisting of hydrogen, halogen, alkyl, nitro, amino, haloalkyl, alkylamino, dialkylamino, acyl, and acyloxy;

Z is selected from the group consisting of hydrogen, alkyl, substituted alkyl, alkenyl, substituted alkenyl, alkynyl, substituted alkynyl, aryl, aralkyl, alkaryl, heteroaryl, heteroaralkyl, a carbohydrate group, —(CH2)—Re, —C(O)-Rg, and —C(O)—(CH2)n—Rh, wherein (a) when each of Ra, Rb, Rc, and Rd are t-butyl, Z cannot be hydrogen; and the other variables are as defined in those specifications, for the treatment of disorders mediated by VCAM-1, and inflammatory and cardiovascular disorders.

WO 01/70757 filed by AtheroGenics, Inc. and published on Sep. 27, 2001, describes the use of certain thioethers of the following formula, and pharmaceutically acceptable salts thereof:

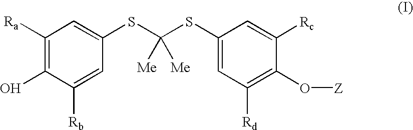

wherein

a) Ra, Rb, Rc, and Rd are independently any group that does not adversely affect the desired properties of the molecule, including hydrogen, alkyl, substituted alkyl, aryl, substituted aryl, heteroaryl, substituted heteroaryl, alkaryl, substituted alkaryl, aralkyl, or substituted aralkyl; and

b) Z is (i) a substituted or unsubstituted carbohydrate, (ii) a substituted or unsubstituted alditol, (iii) C1-10alkyl or substituted C1-10alkyl, terminated by sulfonic acid, (iv) C1-10alkyl or substituted C1-10alkyl, terminated by phosphonic acid, (v) substituted or unsubstituted C1-10alkyl-O—C(O)—C1-10alkyl, (vi) straight chained polyhydroxylated C3-10 alkyl; (vii) —(CR2)1-6-COOH, wherein R is independently hydrogen, halo, amino, or hydroxy, and

wherein at least one of the R substituents is not hydrogen; or (viii) -(CR2)1-6-X, wherein X is aryl, heteroaryl, or heterocycle, and R is independently hydrogen, halo, amino, or hydroxy.

Meng et al., discloses a series of phenolic compounds that has been discovered as potent inhibitors of TNF-α-inducible expression of vascular cell adhesion molecule-1 (VCAM-1) with concurrent antioxidant and lipid-modulating properties. The compounds disclosed have demonstrated efficacies in animal models of atherosclerosis and hyperlipidemia. (Novel Phenolic Antioxidants As Multifunctional Inhibitors Of inducible VCAM-1 Expression For Use In Atherosclerosis, Bioorganic & Medl Chem Ltrs. 12(18), 2545-2548, 2002).

Sundell et al., discloses a novel metabolically stable phenolic antioxidant compound derived from probucol. ([4-[[1-[[3,5-bis(1,1-dimethylethyl)-4-hydroxypehenyl]thio]-1-methylethyl]thio]2,6-bis(1,1-dimethylethyl) phenoxy]acetic acid) inhibits TNF-α-stimulated endothelial expression of VCAM-1 and MCP-1, two redox-sensitive inflammatory genes critical for the recruitment of leukocytes to joints in rheumatoid arthritis (RA), to a greater extent than ICAM-1. (AGIX-4207: A Novel Antioxidant And Anti-Inflammatory Compound Inhibits Progression Of Collagen II Arthritis In The Rat, FASEB Journal Vol. 16, November 4, PP. A182, Mar. 20, 2002. Apr. 20-24, 2002, Annual Meeting of the Professional Research Scientists on Experimental Biology, ISSN 0892-6638).

It is an object of the present invention to provide a method and composition to increase cardiovascular health in mammals.

### BRIEF SUMMARY OF THE INVENTION

It has been surprisingly discovered that the compounds of Formula I below have a direct effect on the lumen diameter of coronary blood vessels, and thus can be used, in one embodiment, to reverse cardiovascular disease. This is a stunning result of human clinical trials and could not have been predicted in advance of these trials.

As an illustration, a 305-patient clinical trial was performed that compared three doses of compound A (70 mg, 140 mg and 280 mg once a day), given for six weeks, to placebo and probucol (500 mg given twice a day), a drug that has been shown to prevent restenosis. The primary endpoint of the trial was the size of the lumenal area (coronary artery opening), as measured by intravascular ultrasound (IVUS), six months after angioplasty. The experimental results showed that the study met its primary endpoint, with mean minimal luminal areas of: 2.66 mm2 (placebo); 3.69 mm2 (probucol); 2.75 mm2 (70 mg), 3.17 mm2 (140 mg) and 3.36 mm2 (280 mg) (p<0.05 for both the Compound A dose response and for 280 mg Compound A vs. placebo). Angiographic restenosis was also assessed using a standard definition of restenosis as measured by quantitative coronary angiography (QCA). Rates of angiographic restenosis in stented arteries were 37.5 percent for placebo, 25.5 percent for probucol, and 26.0 percent in the combined Compound A arms. This yielded a restenosis rate reduction of 32 percent and 31 percent by probucol and Compound A, respectively. Importantly, an early direct benefit on coronary artery disease was evident at two weeks as shown by a dose response improvement (p<0.05) of the luminal area at the site of angioplasty for patients who received Compound A. This direct benefit was maintained at the angioplasty site at the six-month follow-up, as measured by repeat angiography. An IVUS analysis of reference vessels (blood vessels of coronary arteries that were not targets of angioplasty procedures) was also carried out. The data indicated lumen volumes increased for patients who received either of the top two doses of Compound A. In contrast, patients on placebo had decreased lumen volumes, consistent with the expected progression of atherosclerosis. These lumen volume changes were measured as: −5.3 mm3 for placebo, −0.2 mm3 for probucol, −2.4 mm3 for Compound A 70 mg, +3.5 mm3 for Compound A 140 mg, and +1.8 mm3 for Compound A 280 mg.

In a first embodiment, therefore, the invention is a method to increase the lumen diameter of a blood vessel that includes administering an effective lumen diameter decreasing amount of a compound of Formula I. In another embodiment, a therapeutic method for preventing, treating or reversing a cardiovascular indication characterized by a decreased lumen diameter is provided. The method comprises administering to a mammal at risk of, or afflicted with, such a cardiovascular indication, a therapeutic amount (i.e., a lumen diameter increasing amount) of a select compound to stop the progression of the disease, reverse the disease, or prevent the disease. In a preferred embodiment, the mammal is a human.

The present invention includes a method of preventing the onset of cardiovascular disease by administering a select compound to a subject who is susceptible to cardiovascular disease characterized by a decreased luminal diameter. The compound can be administered as a prophylactic to a subject who is at risk of cardiovascular disease. In another embodiment, the lumen diameter of a patient is increased prophylactically or prospectively.

A therapeutic method is also provided for treating or preventing cardiovascular pathologies, such as conditions selected from the group consisting of atherosclerosis, thrombosis, myocardial infarction, and stroke.

The methods described herein comprises the systemic or local administration of an effective lumen diameter decreasing amount, of a compound of Formula I

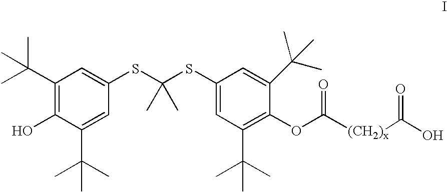

wherein x is selected from 1, 2, 3 or 4;

or a pharmaceutically acceptable salt, ester or prodrug thereof.

Another embodiment of the invention includes the local administration of the compound to an arterial lesion associated with atherosclerosis, and a kit to accomplish said administration.

Another embodiment of the present invention includes employing the compounds of the invention with other compounds having complementary effects or complementary modes of action.. Compounds of the present invention can be administered in combination with a drug that lowers cholesterol via a different biological pathway, to provide augmented results. For example, ileal bile acid transporter (IBAT) inhibitors frequently lower LDL lipoprotein but also lower HDL lipoprotein. A therapeutic combination of an IBAT inhibitor and a compound of the present invention will, when dosages are optimally adjusted, lower LDL yet maintain or raise HDL.

### BRIEF DESCRIPTION OF THE DRAWINGS

FIG. 1 is a bar chart graph comparing QT interval of placebo, probucol (500 mg twice a day) and Compound A (monosuccinic acid ester of probucol) (70, 140 and 280 mg, once daily).

FIG. 2 is a bar chart graph of minimal lumen area as assessed by intravascular ultrasound (IVUS), both pre- and post percutaneous coronary intervention (PCI).

FIG. 3 is a bar chart graph of minimal lumen area assessed upon follow-up.

FIG. 4 is a bar chart graph depicts quantitative coronary angiography (QCA) procedural failure and in-stent restenosis.

FIG. 5 depicts reference segment lumen volume at a non-PCI site.

FIG. 6 depicts lumen volume change between placebo, probucol and Compound A showing surprising results of Compound A to increase lumen volume at a non-PCI site.

### DETAILED DESCRIPTION OF THE INVENTION

It has been surprisingly discovered that the compounds of Formula I below have a direct effect on lumen diameter of blood vessels, and thus can be used, in one embodiment, to reverse cardiovascular disease. This is a stunning result of human clinical trials and could not have been predicted in advance of these trials.

The present invention thus includes a therapeutic method for increasing the lumen diameter of a blood vessel that includes administering a lumen diameter increasing amount of a compound of Formula I. In another embodiment, the method includes preventing, treating, or reversing, a cardiovascular indication characterized by a decreased lumen diameter. The method comprises administering to a mammal at risk of, or afflicted with, said cardiovascular indication, a therapeutic amount of a select compound to stop the progression of the disease, reverse the disease, or prevent the disease.

Another embodiment of the invention comprises the local administration of the compound to an arterial lesion associated with atherosclerosis, and a kit to accomplish said administration.

Another embodiment of the present invention comprises employing the compounds of the invention with other compounds having complementary effects or complementary modes of action. Compounds of the present invention can be administered in combination with a drug that lowers cholesterol via the same or a different biological pathway, to provide augmented results. For example, ileal bile acid transporter (IBAT) inhibitors frequently lower LDL lipoprotein but also lower HDL lipoprotein. A therapeutic combination of an IBAT inhibitor and a compound of the present invention will, when dosages are optimally adjusted, lower LDL yet maintain or raise HDL.

A therapeutic method is provided for treating or preventing cardiovascular pathologies, such as conditions selected from the group consisting of atherosclerosis, thrombosis, myocardial infarction, and stroke. The method comprises the systemic or local administration of an amount of a compound of Formula I

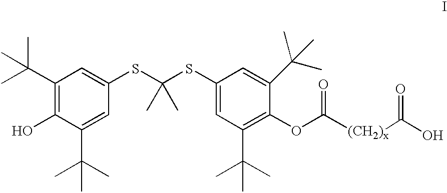

wherein x is selected from 1, 2, 3 or 4;

or a pharmaceutically acceptable salt, ester or prodrug thereof.

A particular compound of Formula I is Compound A represented by

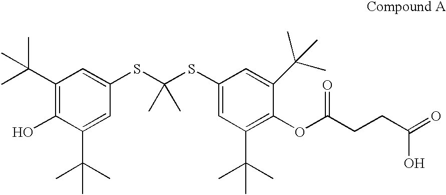

or its pharmaceutically acceptable salt, ester or prodrug thereof.

I. Definitions

The term “pharmaceutically acceptable salts” refer to salts or complexes that retain the desired biological activity of the compounds of the present invention and exhibit minimal undesired toxicological effects. Nonlimiting examples of such salts are (a) acid addition salts formed with inorganic acids (for example, hydrochloric acid, hydrobromic acid, sulfuric acid, phosphoric acid, nitric acid, and the like), and salts formed with organic acids such as acetic acid, oxalic acid, tartaric acid, succinic acid, malic acid, ascorbic acid, benzoic acid, tannic acid, pamoic acid, alginic acid, polyglutamic acid, naphthalenesulfonic acid, naphthalenedisulfonic acid, and polygalcturonic acid; (b) base addition salts formed with metal cations such as zinc, calcium, bismuth, barium, magnesium, aluminum, copper, cobalt, nickel, cadmium, sodium, potassium, and the like, or with a cation formed from ammonia, N,N-dibenzylethylenediamine, D-glucosamine, tetraethylammonium, or ethylenediamine; or (c) combinations of (a) and (b); e.g., a zinc tannate salt or the like. Also included in this definition are pharmaceutically acceptable quaternary salts known by those skilled in the art, which specifically include the quaternary ammonium salt of the formula —NR+A—, wherein R is as defined above and A is a counterion, including chloride, bromide, iodide, —O-alkyl, toluenesulfonate, methylsulfonate, sulfonate, phosphate, or carboxylate (such as benzoate, succinate, acetate, glycolate, maleate, malate, citrate, tartrate, ascorbate, benzoate, cinnamoate, mandeloate, benzyloate, and diphenylacetate).

In cases where compounds are sufficiently basic or acidic to form stable nontoxic acid or base salts, administration of the compounds as salts may be appropriate. Examples of pharmaceutically acceptable salts are organic acid addition salts formed with acids which form a physiological acceptable anion, for example, tosylate, methanesulfonate, acetate, citrate, malonate, tartarate, succinate, benzoate, ascorbate, α-ketoglutarate, and α-glycerophosphate. Suitable inorganic salts may also be formed, including, sulfate, nitrate, bicarbonate, and carbonate salts.

Pharmaceutically acceptable salts may be obtained using standard procedures well known in the art, for example by reacting a sufficiently basic compound such as an amine with a suitable acid affording a physiologically acceptable anion. Alkali metal (for example, sodium, potassium or lithium) or alkaline earth metal (for example calcium) salts of carboxylic acids can also be made.

II. Stereoisomerism and Polymorphism

It is appreciated that compounds of the present invention having a chiral center may exist in and be isolated in optically active and racemic forms. Some compounds may exhibit polymorphism. It is to be understood that the present invention encompasses any racemic, optically-active, polymorphic, or stereoisomeric form, or mixtures thereof, of a compound of the invention, which possess the useful properties described herein, it being well known in the art how to prepare optically active forms and how to determine antiproliferative activity using the standard tests described herein, or using other similar tests which are well known in the art. Examples of methods that can be used to obtain optical isomers of the compounds of the present invention include the following.

i) physical separation of crystals—a technique whereby macroscopic crystals of the individual enantiomers are manually separated. This technique can be used if crystals of the separate enantiomers exist, i.e., the material is a conglomerate, and the crystals are visually distinct;

ii) simultaneous crystallization—a technique whereby the individual enantiomers are separately crystallized from a solution of the racemate, possible only if the latter is a conglomerate in the solid state;

iii) enzymatic resolutions—a technique whereby partial or complete separation of a racemate by virtue of differing rates of reaction for the enantiomers with an enzyme

iv) enzymatic asymmetric synthesis—a synthetic technique whereby at least one step of the synthesis uses an enzymatic reaction to obtain an enatiomerically pure or enriched synthetic precursor of the desired enantiomer;

v) chemical asymmetric synthesis—a synthetic technique whereby the desired enantiomer is synthesized from an achiral precursor under conditions that produce assymetry (i.e., chirality) in the product, which may be achieved using chrial catalysts or chiral auxiliaries;

vi) diastereomer separations—a technique whereby a racemic compound is reacted with an enantiomerically pure reagent (the chiral auxiliary) that converts the individual enantiomers to diastereomers. The resulting diastereomers are then separated by chromatography or crystallization by virtue of their now more distinct structural differences and the chiral auxiliary later removed to obtain the desired enantiomer;

vii) first- and second-order asymmetric transformations—a technique whereby diastereomers from the racemate equilibrate to yield a preponderance in solution of the diastereomer from the desired enantiomer or where preferential crystallization of the diastereomer from the desired enantiomer perturbs the equilibrium such that eventually in principle all the material is converted to the crystalline diastereomer from the desired enantiomer. The desired enantiomer is then released from the diastereomer;

viii) kinetic resolutions—this technique refers to the achievement of partial or complete resolution of a racemate (or of a further resolution of a partially resolved compound) by virtue of unequal reaction rates of the enantiomers with a chiral, non-racemic reagent or catalyst under kinetic conditions;

ix) enantiospecific synthesis from non-racemic precursors—a synthetic technique whereby the desired enantiomer is obtained from non-chiral starting materials and where the stereochemical integrity is not or is only minimally compromised over the course of the synthesis;

x) chiral liquid chromatography—a technique whereby the enantiomers of a racemate are separated in a liquid mobile phase by virtue of their differing interactions with a stationary phase. The stationary phase can be made of chiral material or the mobile phase can contain an additional chiral material to provoke the differing interactions;

xi) chiral gas chromatography—a technique whereby the racemate is volatilized and enantiomers are separated by virtue of their differing interactions in the gaseous mobile phase with a column containing a fixed non-racemic chiral adsorbent phase;

xii) extraction with chiral solvents—a technique whereby the enantiomers are separated by virtue of preferential dissolution of one enantiomer into a particular chiral solvent;

xiii) transport across chiral membranes—a technique whereby a racemate is placed in contact with a thin membrane barrier. The barrier typically separates two miscible fluids, one containing the racemate, and a driving force such as concentration or pressure differential causes preferential transport across the membrane barrier. Separation occurs as a result of the non-racemic chiral nature of the membrane which allows only one enantiomer of the racemate to pass through.

III. Active Compounds

It has been discovered that the compounds of formula I increase the lumen diameter of coronary blood vessels.

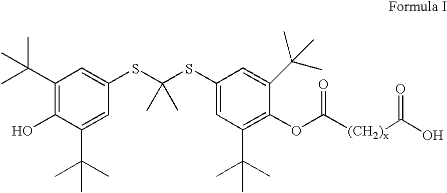

wherein x is selected from 1, 2, 3 or 4.

In a preferred embodiment, the compound is:

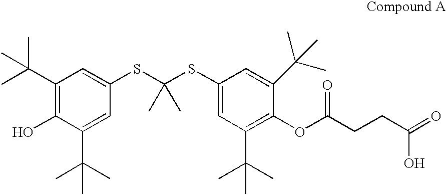

IV. Pharmaceutical Compositions

While it may be possible for the compounds of the invention to be administered as the raw chemical, it is preferable to provide them as a pharmaceutical composition, in an effective lumen diameter increasing amount. According to a further aspect, the present invention provides a pharmaceutical composition comprising a compound of the invention or a pharmaceutically acceptable salt or solvate thereof, together with one or more pharmaceutically acceptable carriers thereof and optionally one or more other therapeutic ingredients for any of the indications specified herein. The carrier(s) must be acceptable in the sense of being compatible with the other ingredients of the formulation and not deleterious to the recipient thereof.

The formulations include those suitable for oral, parenteral (including subcutaneous, intradermal, intramuscular, intravenous and intraarticular), rectal and topical (including dermal, buccal, sublingual and intraocular) administration although the most suitable route may depend upon for example the condition and disorder of the recipient. The formulations may conveniently be presented in unit dosage form and may be prepared by any of the methods well known in the art of pharmacy. All methods include the step of bringing into association a compound of the invention or a pharmaceutically acceptable salt or solvate thereof (“active ingredient”) with the carrier which constitutes one or more accessory ingredients. In general, the formulations are prepared by uniformly and intimately bringing into association the active ingredient with liquid carriers or finely divided solid carriers or both and then, if necessary, shaping the product into the desired formulation.

Formulations of the present invention suitable for oral administration may be presented as discrete units such as capsules, cachets or tablets each containing a predetermined amount of the active ingredient; as a powder or granules; as a solution or a suspension in an aqueous liquid or a non-aqueous liquid; or as an oil-in-water liquid emulsion or a water-in-oil liquid emulsion. The active ingredient may also be presented as a bolus, electuary or paste.

A tablet may be made by compression or molding, optionally with one or more accessory ingredients. Compressed tablets may be prepared by compressing in a suitable machine the active ingredient in a free-flowing form such as a powder or granules, optionally mixed with a binder, lubricant, inert diluent, lubricating, surface active or dispersing agent. Molded tablets may be made by molding in a suitable machine a mixture of the powdered compound moistened with an inert liquid diluent. The tablets may optionally be coated or scored and may be formulated so as to provide slow or controlled release of the active ingredient therein.

Formulations for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example sealed ampuls and vials, and may be stored in a freeze-dried (lyophilized) condition requiring only the addition of the sterile liquid carrier, for example, saline, water-for-injection, immediately prior to use. Extemporaneous injection solutions and suspensions may be prepared from sterile powders, granules and tablets of the kind previously described.

Formulations for rectal administration may be presented as a suppository with the usual carriers such as cocoa butter or polyethylene glycol.

Formulations for topical administration in the mouth, for example buccally or sublingually, include lozenges comprising the active ingredient in a flavored basis such as sucrose and acacia or tragacanth, and pastilles comprising the active ingredient in a basis such as gelatin and glycerin or sucrose and acacia.

Preferred unit dosage formulations are those containing an effective dose, as herein below recited, or an appropriate fraction thereof, of the active ingredient.

It should be understood that in addition to the ingredients particularly mentioned above, the formulations of this invention may include other agents conventional in the art having regard to the type of formulation in question, for example those suitable for oral administration may include flavoring agents.

The compounds of the invention may be administered orally or by injection (intravenous or subcutaneous). The precise amount of compound administered to a patient will be the responsibility of the attendant physician. However, the dose employed will depend on a number of factors, including the age and sex of the patient, the precise disorder being treated, and its severity. Also, the route of administration may vary depending on the condition and its severity.

The compounds of the invention may be administered orally or via injection in a lumen diameter-increasing amount. The dose range for humans is generally from 0.005 mg to 10 g/day. Tablets or other forms of presentation provided in discrete units may conveniently contain an amount of compound of the invention which is effective at such dosage or as a multiple of the same, for instance, units containing 5 mg to 500 mg, usually around 10 mg to 200 mg.

The compounds of the present invention can also be administered via a catheter or stent, for example, by use of an intraluminal stent. Although stents are commonly used as part of an angioplasty procedure, intraluminal stents can be used to maintain or control any bodily luminal opening. The compound of the present invention could be used alone or as part of a composition allowing for a controlled release of the therapeutically active compound. The compounds could be coated on the stent or made a part of the stent. They may be layered so as to provide limited release of the active compound, or used in any manner known in the art as disclosed in U.S. patent application Nos. 20010029660 and 20010032014.

Animals, particularly mammal, and more particularly, humans, equine, canine, and bovine can be treated for any of the conditions described herein by administering to the subject an effective amount of one or more of the above-identified compounds or a pharmaceutically acceptable prodrug or salt thereof in a pharmaceutically acceptable carrier or diluent. Any appropriate route can be used to administer the active materials, for example, orally, parenterally, intravenously, intradermally, subcutaneously or topically.

The active compound is included in the pharmaceutically acceptable carrier or diluent in an amount sufficient to deliver to a patient a therapeutically effective amount without causing serious toxic effects in the patient treated. A preferred dose of the active compound for all of the above-mentioned conditions is in the range from about 0.1 to 500 mg/kg, preferably 1 to 100 mg/kg per day. The effective dosage range of the pharmaceutically acceptable prodrugs can be calculated based on the weight of the parent compound to be delivered. If the derivative exhibits activity in itself, the effective dosage can be estimated as above using the weight of the derivative, or by other means known to those skilled in the art.

For systemic administration, the compound is conveniently administered in any suitable unit dosage form, including but not limited to one containing 1 to 3000 mg, preferably 5 to 500 mg of active ingredient per unit dosage form. An oral dosage of 25-250 mg is usually convenient. The active ingredient should be administered to achieve peak plasma concentrations of the active compound of about 0.1 to 100 mM, preferably about 1 -10 mM. This may be achieved, for example, by the intravenous injection of a solution or formulation of the active ingredient, optionally in saline, or an aqueous medium or administered as a bolus of the active ingredient.

The concentration of active compound in the drug composition will depend on absorption, distribution, inactivation and excretion rates of the drug as well as other factors known to those of skill in the art. It is to be noted that dosage values will also vary with the severity of the condition to be alleviated. It is to be further understood that for any particular subject, specific dosage regimens should be adjusted over time according to the individual need and the professional judgment of the person administering or supervising the administration of the compositions, and that the concentration ranges set forth herein are exemplary only and are not intended to limit the scope or practice of the claimed composition. The active ingredient may be administered at once, or may be divided into a number of smaller doses to be administered at varying intervals of time.

Oral compositions will generally include an inert diluent or an edible carrier. They may be enclosed in gelatin capsules or compressed into tablets. For the purpose of oral therapeutic administration, the active compound can be incorporated with excipients and used in the form of tablets, troches or capsules. Pharmaceutically compatible binding agents, and/or adjuvant materials can be included as part of the composition.

The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring. When the dosage unit form is a capsule, it can contain, in addition to material of the above type, a liquid carrier such as a fatty oil. In addition, dosage unit forms can contain various other materials which modify the physical form of the dosage unit, for example, coatings of sugar, shellac, or other enteric agents.

The active compound or pharmaceutically acceptable salt or derivative thereof can be administered as a component of an elixir, suspension, syrup, wafer, chewing gum or the like. A syrup may contain, in addition to the active compounds, sucrose as a sweetening agent and certain preservatives, dyes and colorings and flavors.

The active compound or pharmaceutically acceptable prodrugs or salts thereof can also be administered with other active materials that do not impair the desired action, or with materials that supplement the desired action, such as antibiotics, anti-fungals, anti-inflammatories, or antiviral compounds. The active compounds can be administered with lipid lowering agents such as probucol and nicotinic acid; platelet aggregation inhibitors such as aspirin; antithrombotic agents such as coumadin; calcium channel blockers such as varapamil, diltiazem, and nifedipine; angiotensin converting enzyme (ACE) inhibitors such as captopril and enalopril, and β-blockers such as propanalol, terbutalol, and labetalol. The compounds can also be administered in combination with nonsteroidal antiinflammatories such as ibuprofen, indomethacin, aspirin, fenoprofen, mefenamic acid, flufenamic acid, sulindac. The compound can also be administered with corticosteriods.

Solutions or suspensions used for parenteral, intradermal, subcutaneous, or topical application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. The parental preparation can be enclosed in ampoules, disposable syringes or multiple dose vials made of glass or plastic.

Suitable vehicles or carriers for topical application are known, and include lotions, suspensions, ointments, creams, gels, tinctures, sprays, powders, pastes, slow-release transdermal patches, aerosols for asthma, and suppositories for application to rectal, vaginal, nasal or oral mucosa.

Thickening agents, emollients and stabilizers can be used to prepare topical compositions. Examples of thickening agents include petrolatum, beeswax, xanthan gum or polyethylene glycol, humectants such as sorbitol, emollients such as mineral oil, lanolin and its derivatives, or squalene. A number of solutions and ointments are commercially available.

Natural or artificial flavorings or sweeteners can be added to enhance the taste of topical preparations applied for local effect to mucosal surfaces. Inert dyes or colors can be added, particularly in the case of preparations designed for application to oral mucosal surfaces.

The active compounds can be prepared with carriers that protect the compound against rapid release, such as a controlled release formulation, including implants and microencapsulated delivery systems. Biodegradable, biocompatible polymers can be used, such as ethylene vinyl acetate, polyanhydrides, polyglycolic acid, collagen, polyorthoesters and polylacetic acid. Many methods for the preparation of such formulations are patented or generally known to those skilled in the art.

If administered intravenously, preferred carriers are physiological saline or phosphate buffered saline (PBS).

The active compound can also be administered through a transdermal patch. Methods for preparing transdermal patches are known to those skilled in the art. For example, see Brown, L., and Langer, R., Transdermal Delivery of Drugs, Annual Review of Medicine, 39:221-229 (1988).

In another embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters and polylacetic acid. Methods for preparation of such formulations will be apparent to those skilled in the art. The materials can also be obtained commercially from Alza Corporation and Nova Pharmaceuticals, Inc. Liposomal suspensions may also be pharmaceutically acceptable carriers. These may be prepared according to methods known to those skilled in the art, for example, as described in U.S. Pat. No. 4,522,811. For example, liposome formulations may be prepared by dissolving appropriate lipid(s) (such as stearoyl phosphatidyl ethanolamine, stearoyl phosphatidyl choline, arachadoyl phosphatidyl choline, and cholesterol) in an inorganic solvent that is then evaporated, leaving behind a thin film of dried lipid on the surface of the container. An aqueous solution of the active compound or its monophosphate, diphosphate, and/or triphosphate derivatives are then introduced into the container. The container is then swirled by hand to free lipid material from the sides of the container and to disperse lipid aggregates, thereby forming the liposomal suspension.

V. Combination or Alternation Therapy

The compounds of the present invention can be combined with other biologically active compounds to achieve any desired therapeutic goal. For example, through dosage adjustment and medical monitoring, the individual dosages of the therapeutic compounds used in the combinations of the present invention will be lower than are typical for dosages of the therapeutic compounds when used in monotherapy. The dosage lowering will provide advantages including reduction of side effects of the individual therapeutic compounds when compared to the monotherapy. In addition, fewer side effects of the combination therapy compared with the monotherapies will lead to greater patient compliance with therapy regimens.

Another use of the present invention will be in combinations having complementary effects or complementary modes of action. Compounds of the present invention can be administered in combination with a drug that lowers cholesterol via a different biological pathway, to provide augmented results. For example, ileal bile acid transporter (IBAT) inhibitors frequently lower LDL lipoprotein but also lower HDL lipoprotein. A therapeutic combination of an IBAT inhibitor and a compound of the present invention will, when dosages are optimally adjusted, lower LDL yet maintain or raise HDL.

Compounds useful for combining with the compounds of the present invention encompass a wide range of therapeutic compounds. IBAT inhibitors, for example, are useful in the present invention, and are disclosed in patent application Nos. PCT/US95/10863 and in PCT/US97/04076. Still further IBAT inhibitors useful in the present invention are described in U.S. application Ser. No. 08/816,065. More IBAT inhibitor compounds useful in the present invention are described in WO 98/40375, and WO 00/38725. Additional IBAT inhibitor compounds useful in the present invention are described in U.S. application Ser. No. 08/816,065 and U.S. Pat. Nos. 6,263,342, 6,420,417, 6,387,924, and 6,107,494.

In another aspect, the second cholesterol lowering agent is a statin. The combination of the HDLc enhancing drug with a statin creates a synergistic or augmented lowering of serum cholesterol, because statins lower cholesterol by a different mechanism, i.e., by inhibiting of 3-hydroxy-3-methylglutaryl coenzyme A (HMG CoA) reductase, a key enzyme in the cholesterol biosynthetic pathway. The statins decrease liver cholesterol biosynthesis, which increases the production of LDL receptors thereby decreasing plasma total and LDL cholesterol (Grundy, S. M. New Engl. J. Med. 319, 24 (1988); Endo, A. J. Lipid Res. 33, 1569 (1992)). Depending on the agent and the dose used, statins may decrease plasma triglyceride levels and may increase HDLc. Currently the statins on the market are lovastatin (Merck), simvastatin (Merck), pravastatin (Sankyo and Squibb) and fluvastatin (Sandoz). A fifth statin, atorvastatin (Parke-Davis/Pfizer), is the most recent entrant into the statin market. Any of these or other statins can be used in combination to functionality improve the drug of the present invention.

The following list discloses these preferred statins and their preferred dosage ranges.

TABLE 1

| | | Dosage | Normal | |
|---|---|---|---|---|
| | | range | dose | Patent |
| | Trade name | (mg/d) | (mg/d) | Reference |
| Fungal derivatives | | | | |
| lovastatin | Mevacor | 10-80 | 20-40 | 4,231,938 |
| pravastatin | Pravachol | 10-40 | 20-40 | 4,346,227 |
| simvastatin | Zocor | 5-40 | 5-10 | 4,739,073 |
| Synthetic compound | | | | |
| Fluvastatin | Lescol | 20-80 | 20-40 | 4,739,073 |

The following list describes the chemical formula of some preferred statins:

lovastatin: [1S[1a(R),3 alpha,7 beta,8 beta (2S,4S),8a beta]]-1,2,3,7,8,8a-hexahydro-3,7-dimethyl-8-[2-(tetrahydro-4-hydroxy-6-oxo-2H-pyran-2-yl)ethyl]-1-maphthalenyl-2-methylbutanoate

pravastatin sodium: 1-Naphthalene-heptanoic acid, 1,2,6,7,8a-hexahydro- beta, delta ,6-trihydroxy-2-methyl-8-(2-ethyl-1-oxybutoxy)-1-, monosodium salt [1S-[1alpha(beta s, delta S),2 alpha ,6 alpha ,8 beta (R),8a alpha

simvastatin: butanoic acid, 2,2-dimethyl-,1,2,3,7,8,8a-hexahydro-3,7-dimethyl-8-[2tetrahydro-4-hydroxy-6-oxo-2H-pyran-2-yl)ethyl]-1-napthalenyl ester[1S-[1 alpha ,3 alpha, 7 beta ,8 beta ,(2S,4S),-8a beta

sodium fluvastatin: [R,S-(E)]-(+/−)-7-[3(4-fluorophenyl)-1-(1-methylethyl)-1H-indol-2-yl]-3,5-dihydroxy-6-heptenoic acid, monosodium salt

Other statins, and references from which their description can be derived, are listed below.

TABLE 2

| STATIN | REFERENCE |
|---|---|
| Atorvastatin | U.S. Pat. No. 5,273,995 |
| Cerivastatin (Baycol) | U.S. Pat. No. 5,177,080 |
| Mevastatin | U.S. Pat. No. 3,983,140 |
| Cerivastatin | U.S. Pat. No. 5,502,199 |
| Velostatin | U.S. Pat. No. 4,448,784 |
| Compactin | U.S. Pat. No. 4,804,770 |
| Dalvastatin | EP 738510 A2 |
| Fluindostatin | EP 363934 A1 |
| Dihydorcompactin | U.S. Pat. No. 4,450,171 |

Other statins include rivastatin, SDZ-63,370 (Sandoz), CI-981 (W-L). HR-780, L-645,164, CL-274,471, alpha-, beta-, and gamma-tocotrienol, (3R,5S,6E)-9,9-bis(4-fluoro-phenyl)-3,5-dihydroxy-8-(1-methyl-1H-tetrazol-5-yl)-6,8-nonadienoic acid, L-arginine salt, (S)-4-[[2-[4-(4-fluorophenyl)-5-methyl-2-(1-methylethyl)-6-phenyl-3-pyridinyl]ethenyl]-hydroxyphosphinyl]-3-hydroxybutanoic acid, disodium salt, BB-476, (British Biotechnology), dihydrocompactin, [4R-[4 alpha ,6 beta (E)]]-6-[2-[5-(4-fluorophenyl)-3-(1- methylethyl)-1-(2-pyridinyl)-1H-pyrazol-4-yl]ethenyl]tetrahydro-4-hydroxy-2H-pyran-2-one, and 1H-pyrrole-1-heptanoic acid, 2-(4-fluorophenyl)-beta,delta-dihydroxy-5-(1-methylethyl)-3-phenyl-4-[(phenylamino)carbonyl]calcium salt[R-(R*,R*)].

However, the invention should not be considered to be limited to the foregoing statins. Naturally occurring statins are derivatives of fungi metabolites (ML-236B/compactin/monocalin K) isolated from Pythium ultimum, Monacus ruber, Penicillium citrinum, Penicillium brevicompactum and Aspergillus terreus, though as shown above they can be prepared synthetically as well. Statin derivatives are well known in the literature and can be prepared by methods disclosed in U.S. Pat. No. 4,397,786. Other methods are cited in The Peptides: Vol. 5, Analysis, Synthesis, Biology; Academic Press NY (1983); and by Bringmann et al. in Synlett (5), pp. 253-255 (1990).

Thus, the term statin as used herein includes any naturally occurring or synthetic peptide that inhibits 3-hydroxy-3-methylglutaryl coenzyme A (HMG CoA) reductase by competing with 3-hydroxy-3-methylglutaric acid (HMG) CoA for the substrate binding site on HMG-CoA reductase. Assays for determining whether a statin acts through this biological pathway are disclosed in U.S. Pat. No. 4,231,938, column 6, and WO 84/02131 on pages 30-33.

MTP inhibitor compounds useful in the combinations and methods of the present invention comprise a wide variety of structures and functionalities. Some of the MTP inhibitor compounds of particular interest for use in the present invention are disclosed in WO 00/38725 and U.S. Pat. Nos. 6,458,851 and 6,458,850. Descriptions of these therapeutic compounds can be found in Science, 282, Oct. 23, 1998, pp. 751-754.

Cholesterol absorption antagonist compounds useful in the combinations and methods of the present invention comprise a wide variety of structures and functionalities. Some of the cholesterol absorption antagonist compounds of particular interest for use in the present invention are described in U.S. Pat. No. 5,767,115. Further cholesterol absorption antagonist compounds of particular interest for use in the present invention, and methods for making such cholesterol absorption antagonist compounds are described in U.S. Pat. No. 5,631,365.

A number of phytosterols suitable for the combination therapies of the present invention are described by Ling and Jones in “Dietary Phytosterols: A Review of Metabolism, Benefits and Side Effects,” Life Sciences, 57 (3), 195-206 (1995). Without limitation, some phytosterols of particular use in the combination of the present invention are Clofibrate, Fenofibrate, Ciprofibrate, Bezafibrate, Gemfibrozil. The structures of the foregoing compounds can be found in WO 00/38725.

Phytosterols are also referred to generally by Nes (Physiology and Biochemistry of Sterols, American Oil Chemists' Society, Champaign, Ill., 1991, Table 7-2). Especially preferred among the phytosterols for use in the combinations of the present invention are saturated phytosterols or stanols. Additional stanols are also described by Nes (Id.) and are useful in the combination of the present invention. In the combination of the present invention, the phytosterol preferably comprises a stanol. In one preferred embodiment the stanol is campestanol. In another preferred embodiment the stanol is cholestanol. In another preferred embodiment the stanol is clionastanol. In another preferred embodiment the stanol is coprostanol. In another preferred embodiment the stanol is 22,23-dihydrobrassicastanol. In another embodiment the stanol is epicholestanol. In another preferred embodiment the stanol is fucostanol. In another preferred embodiment the stanol is stigmastanol.

In another embodiment the present invention encompasses a therapeutic combination of a compound of the present invention and another HDLc elevating agent. In one aspect, the second HDLc elevating agent can be a CETP inhibitor. Individual CETP inhibitor compounds useful in the present invention are separately described in WO 00/38725. Other individual CETP inhibitor compounds useful in the present invention are separately described in WO 99/14174, EP818448, WO 99/15504, WO 99/14215, WO 98/04528, WO 00/17166 and U.S. Pat. Nos. 6,462,091, 6,458,852, 6,458,850, 6,458,803, and 6,458,849. Other individual CETP inhibitor compounds useful in the present invention are separately described in WO 00/18724, WO 00/18723, and WO 00/18721. Other individual CETP inhibitor compounds useful in the present invention are separately described in WO 98/35937. Particular CETP inhibitors suitable for use in combination with the invention are described in The Discovery of New Cholesteryl Ester Transfer Protein Inhibitors (Sikorski et al., Curr. Opin. Drug Disc. & Dev., 4(5):602-613 (2001)).

In another aspect, the second HDLc elevating agent can be a fibric acid derivative. Fibric acid derivatives useful in the combinations and methods of the present invention comprise a wide variety of structures and functionalities. Particular fibric acid derivatives for the present invention are described in Table 3. The therapeutic compounds of Table 3 can be used in the present invention in a variety of forms, including acid form, salt form, racemates, enantiomers, zwitterions, and tautomers.

TABLE 3

| | | U.S. Patent |
|---|---|---|
| | CAS Registry | Reference for |
| Common Name | Number | Compound Per Se |
| Clofibrate | 637-07-0 | 3,262,850 |
| Fenofibrate | 49562-28-9 | 4,058,552 |
| Ciprofibrate | 52214-84-3 | 3,948,973 |
| Bezafibrate | 41859-67-0 | 3,781,328 |
| Gemfibrozil | 25182-30-1 | 3,674,836 |

In another embodiment the present invention encompasses a therapeutic combination of a compound of the present invention and an antihypertensive agent. Hypertension is defined as persistently high blood pressure. Generally, adults are classified as being hypertensive when systolic blood pressure is persistently above 140 mmHg or when diastolic blood pressure is above 90 mmHg. Long-term risks for cardiovascular mortality increase in a direct relationship with persistent blood pressure. (E. Braunwald, Heart Disease, 5th ed., W. B. Saunders & Co., Philadelphia, 1997, pp. 807-823.) Blood pressure is a function of cardiac output and peripheral resistance of the vascular system and can be represented by the following equation:

BP is CO×PR

wherein BP is blood pressure, CO is cardiac output, and PR is peripheral resistance. (Id., p. 816.) Factors affecting peripheral resistance include obesity and/or functional constriction. Factors affecting cardiac output include venous constriction. Functional constriction of the blood vessels can be caused y a variety of factors including thickening of blood vessel walls resulting in diminishment of the inside diameter of the vessels. Another factor which affects systolic blood pressure is rigidity of the aorta (Id., p. 811.)

Hypertension and atherosclerosis or other hyperlipidemic conditions often coexist in a patient. It is possible that certain hyperlipidemic conditions such as atherosclerosis can have a direct or indirect affect on hypertension. For example, atherosclerosis frequently results in diminishment of the inside diameter of blood vessels. Furthermore, atherosclerosis frequently results in increased rigidity of blood vessels, including the aorta. Both diminished inside diameter of blood vessels and rigidity of blood vessels are factors which contribute to hypertension.

Myocardial infarction is the necrosis of heart muscle cells resulting from oxygen deprivation and is usually cause by an obstruction of the supply of blood to the affected tissue. For example, hyperlipidemia or hypercholesterolemia can cause the formation of atherosclerotic plaques, which can cause obstruction of blood flow and thereby cause myocardial infarction. (Id., pp. 1185-1187.) Another major risk factor for myocardial infarction is hypertension. (Id., p. 815.) In other words, hypertension and hyperlipidemic conditions such as atherosclerosis or hypercholesterolemia work in concert to cause myocardial infarction.

Coronary heart disease is another disease, which is caused or aggravated by multiple factors including hyperlipidemic conditions and hypertension. Control of both hyperlipidemic conditions and hypertension are important to control symptoms or disease progression of coronary heart disease.

Angina pectoris is acute chest pain, which is caused by decreased blood supply to the heart. Decreased blood supply to the heart is known as myocardial ischemia. Angina pectoris can be the result of, for example, stenosis of the aorta, pulmonary stenosis and ventricular hypertrophy. Some antihypertensive agents, for example amlodipine, control angina pectoris by reducing peripheral resistance.

Some antihypertensive agents useful in the present invention are shown in Table 4, without limitation. A wide variety of chemical structures are useful as antihypertensive agents in the combinations of the present invention and the agents can operate by a variety of mechanisms. For example, useful antihypertensive agents can include, without limitation, an adrenergic blocker, a mixed alpha/beta adrenergic blocker, an alpha adrenergic blocker, a beta adrenergic blocker, an adrenergic stimulant, an angiotensin converting enzyme (ACE) inhibitor, an angiotensin II receptor antagonist, a calcium channel blocker, a diuretic, or a vasodilator. Additional hypertensive agents useful in the present invention are described by R. Scott in U.S. Patent Application No. 60/057,276 (priority document for PCT Patent Application No. WO 99/11260).

TABLE 4

| Antihypertensive | | | |
|---|---|---|---|
| Classification | Compound Name | Typical Dosage | |
| adrenergic blocker | Phenoxybenzamine | 1-250 | mg/day |
| adrenergic blocker | Guanadrel | 5-60 | mg/day |
| adrenergic blocker | Guanethidine | | |
| adrenergic blocker | Reserpine | | |
| adrenergic blocker | Terazosin | 0.1-60 | mg/day |
| adrenergic blocker | Prazosin | 0.5-75 | mg/day |
| adrenergic blocker | Polythiazide | 0.25-10 | mg/day |
| adrenergic stimulant | Methyldopa | 100-4000 | mg/day |
| adrenergic stimulant | Methyldopate | 100-4000 | mg/day |
| adrenergic stimulant | Clonidine | 0.1-2.5 | mg/day |
| adrenergic stimulant | Chlorthalidone | 10-50 | mg/day |
| adrenergic blocker | Guanfacine | 0.25-5 | mg/day |
| adrenergic stimulant | Guanabenz | 2-40 | mg/day |
| adrenergic stimulant | Trimethaphan | | |
| alpha/beta adrenergic | Carvedilol | 6-25 | mg bid |
| blocker | | | |
| alpha/beta adrenergic | Labetalol | 10-500 | mg/day |
| blocker | | | |
| beta adrenergic | Propranolol | 10-1000 | mg/day |
| blocker | | | |
| beta adrenergic | Metoprolol | 10-500 | mg/day |
| blocker | | | |
| alpha adrenergic | Doxazosin | 1-16 | mg/day |
| blocker | | | |
| alpha adrenergic | Phentolamine | | |
| blocker | | | |
| angiotensin converting | Quinapril | 1-250 | mg/day |
| enzyme inhibitor | | | |
| angiotensin converting | perindopril erbumine | 1-25 | mg/day |
| enzyme inhibitor | | | |
| angiotensin converting | Ramipril | 0.25-20 | mg/day |
| enzyme inhibitor | | | |
| angiotensin converting | Captopril | 6-50 | mg bid or tid |
| enzyme inhibitor | | | |
| angiotensin converting | Trandolapril | 0.25-25 | mg/day |
| enzyme inhibitor | | | |
| angiotensin converting | Fosinopril | 2-80 | mg/day |
| enzyme inhibitor | | | |
| angiotensin converting | Lisinopril | 1-80 | mg/day |
| enzyme inhibitor | | | |
| angiotensin converting | Moexipril | 1-100 | mg/day |
| enzyme inhibitor | | | |
| angiotensin converting | Enalapril | 2.5040 | mg/day |
| enzyme inhibitor | | | |
| angiotensin converting | Benazepril | 10-80 | mg/day |
| enzyme inhibitor | | | |
| angiotensin II receptor | candesartan cilexetil | 2-32 | mg/day |
| antagonist | | | |
| angiotensin II receptor | Inbesartan | | |
| antagonist | | | |
| angiotensin II receptor | Losartan | 10-100 | mg/day |
| antagonist | | | |
| angiotensin II receptor | Valsartan | 20-600 | mg/day |
| antagonist | | | |
| calcium channel | Verapamil | 100-600 | mg/day |
| blocker | | | |
| calcium channel | Diltiazem | 150-500 | mg/day |
| blocker | | | |
| calcium channel | Nifedipine | 1-200 | mg/day |
| blocker | | | |
| calcium channel | Nimodipine | 5-500 | mg/day |
| blocker | | | |
| calcium channel | Delodipine | | |
| blocker | | | |
| calcium channel | Nicardipine | 1-20 | mg/hr i.v.; |
| blocker | | 5-100 | mg/day oral |
| calcium channel | Isradipine | | |
| blocker | | | |
| calcium channel | Amlodipine | 2-10 | mg/day |
| blocker | | | |
| diuretic | Hydrochlorothiazide | 5-100 | mg/day |
| diuretic | Chlorothiazide | 250-2000 | mg bid or |
| | | | tid |
| diuretic | Furosemide | 5-1000 | mg/day |
| diuretic | Bumetanide | | |
| diuretic | ethacrynic acid | 20-400 | mg/day |
| diuretic | Amiloride | 1-20 | mg/day |
| Diuretic | Triameterene | | |
| Diuretic | Spironolactone | 5-1000 | mg/day |
| Diuretic | Eplerenone | 10-150 | mg/day |
| Vasodilator | Hydralazine | 5-300 | mg/day |
| Vasodilator | Minoxidil | 1-100 | mg/day |
| Vasodilator | Diazoxide | 1-3 | mg/kg |
| Vasodilator | Nitroprusside | | |

Additional calcium channel blockers which are useful in the combinations of the present invention include, without limitation, those shown in Table 5.

TABLE 5

| Compound Name | Reference |
|---|---|
| bepridil | U.S. Pat. No. 3,962,238 or |
| | U.S. Reissue No. 30,577 |
| clentiazem | U.S. Pat. No. 4,567,175 |
| diltiazem | U.S. Pat. No. 3,562,257 |
| fendiline | U.S. Pat. No. 3,262,977 |
| gallopamil | U.S. Pat. No. 3,261,859 |
| mibefradil | U.S. Pat. No. 4,808,605 |
| prenylamine | U.S. Pat. No. 3,152,173 |
| semotiadil | U.S. Pat. No. 4,786,635 |
| terodiline | U.S. Pat. No. 3,371,014 |
| verapamil | U.S. Pat. No. 3,261,859 |
| aranipine | U.S. Pat. No. 4,572,909 |
| bamidipine | U.S. Pat. No. 4,220,649 |
| benidipine | European Patent Application |
| | Publication No. 106,275 |
| cilnidipine | U.S. Pat. No. 4,672,068 |
| efonidipine | U.S. Pat. No. 4,885,284 |
| elgodipine | U.S. Pat. No. 4,962,592 |
| felodipine | U.S. Pat. No. 4,264,611 |
| isradipine | U.S. Pat. No. 4,466,972 |
| lacidipine | U.S. Pat. No. 4,801,599 |
| lercanidipine | U.S. Pat. No. 4,705,797 |
| manidipine | U.S. Pat. No. 4,892,875 |
| nicardipine | U.S. Pat. No. 3,985,758 |
| nifendipine | U.S. Pat. No. 3,485,847 |
| nilvadipine | U.S. Pat. No. 4,338,322 |
| nimodipine | U.S. Pat. No. 3,799,934 |
| nisoldipine | U.S. Pat. No. 4,154,839 |
| nitrendipine | U.S. Pat. No. 3,799,934 |
| cinnarizine | U.S. Pat. No. 2,882,271 |
| flunarizine | U.S. Pat. No. 3,773,939 |
| lidoflazine | U.S. Pat. No. 3,267,104 |
| lomerizine | U.S. Pat. No. 4,663,325 |
| Bencyclane | Hungarian Patent No. 151,865 |
| Etafenone | German Patent No. 1,265,758 |
| Perhexiline | British Patent No. 1,025,578 |

Additional ACE inhibitors which are useful in the combinations of the present invention include, without limitation, those shown in Table 6.

TABLE 6

| Compound Name | Reference |
|---|---|
| alacepril | U.S. Pat. No. 4,248,883 |
| benazepril | U.S. Pat. No. 4,410,520 |
| captopril | U.S. Pat. Nos. 4,046,889 and |
| | 4,105,776 |
| ceronapril | U.S. Pat. No. 4,452,790 |
| delapril | U.S. Pat. No. 4,385,051 |
| enalapril | U.S. Pat. No. 4,374,829 |
| fosinopril | U.S. Pat. No. 4,337,201 |
| imadapril | U.S. Pat. No. 4,508,727 |
| lisinopril | U.S. Pat. No. 4,555,502 |
| moveltopril | Belgian Patent No. 893,553 |
| perindopril | U.S. Pat. No. 4,508,729 |
| quinapril | U.S. Pat. No. 4,344,949 |
| ramipril | U.S. Pat. No. 4,587,258 |
| Spirapril | U.S. Pat. No. 4,470,972 |
| Temocapril | U.S. Pat. No. 4,699,905 |
| Trandolapril | U.S. Pat. No. 4,933,361 |

Additional beta adrenergic blockers which are useful in the combinations of the present invention include, without limitation, those shown in Table 7.

TABLE 7

| Compound Name | Reference |
|---|---|
| acebutolol | U.S. Pat. No. 3,857,952 |
| alprenolol | Netherlands Patent Application No. |
| | 6,605,692 |
| amosulalol | U.S. Pat. No. 4,217,305 |
| arotinolol | U.S. Pat. No. 3,932,400 |
| atenolol | U.S. Pat. No. 3,663,607 or |
| | U.S. Pat. No. 3,836,671 |
| befunolol | U.S. Pat. No. 3,853,923 |
| betaxolol | U.S. Pat. No. 4,252,984 |
| bevantolol | U.S. Pat. No. 3,857,981 |
| bisoprolol | U.S. Pat. No. 4,171,370 |
| bopindolol | U.S. Pat. No. 4,340,641 |
| bucumolol | U.S. Pat. No. 3,663,570 |
| bufetolol | U.S. Pat. No. 3,723,476 |
| bufuralol | U.S. Pat. No. 3,929,836 |
| bunitrolol | U.S. Pat. Nos. 3,940,489 and |
| | U.S. Pat. No. 3,961,071 |
| buprandolol | U.S. Pat. No. 3,309,406 |
| butiridine | French Patent No. 1,390,056 |
| hydrochloride | |
| butofilolol | U.S. Pat. No. 4,252,825 |
| carazolol | German Patent No. 2,240,599 |
| carteolol | U.S. Pat. No. 3,910,924 |
| carvedilol | U.S. Pat. No. 4,503,067 |
| celiprolol | U.S. Pat. No. 4,034,009 |
| cetamolol | U.S. Pat. No. 4,059,622 |
| cloranolol | German Patent No. 2,213,044 |
| dilevalol | Clifton et al., Journal of Medicinal |
| | Chemistry, 1982 25, 670 |
| epanolol | European Patent Publication |
| | Application No. 41,491 |
| indenolol | U.S. Pat. No. 4,045,482 |
| labetalol | U.S. Pat. No. 4,012,444 |
| levobunolol | U.S. Pat. No. 4,463,176 |
| mepindolol | Seeman et al., Helv. Chim. Acta, |
| | 1971, 54, 241 |
| metipranolol | Czechoslovakian Patent Application |
| | No. 128,471 |
| metoprolol | U.S. Pat. No. 3,873,600 |
| moprolol | U.S. Pat. No. 3,501,769 |
| nadolol | U.S. Pat. No. 3,935,267 |
| nadoxolol | U.S. Pat. No. 3,819,702 |
| nebivalol | U.S. Pat. No. 4,654,362 |
| nipradilol | U.S. Pat. No. 4,394,382 |
| oxprenolol | British Patent No. 1,077,603 |
| perbutolol | U.S. Pat. No. 3,551,493 |
| pindolol | Swiss Patent Nos. 469,002 and |
| | Swiss Patent Nos. 472,404 |
| practolol | U.S. Pat. No. 3,408,387 |
| pronethalol | British Patent No. 909,357 |
| propranolol | U.S. Pat. Nos. 3,337,628 and |
| | U.S. Pat. Nos. 3,520,919 |
| sotalol | Uloth et al., Journal of Medicinal |
| | Chemistry, 1966, 9, 88 |
| sufinalol | German Patent No. 2,728,641 |
| talindol | U.S. Pat. Nos. 3,935,259 and |
| | U.S. Pat. Nos. 4,038,313 |
| tertatolol | U.S. Pat. No. 3,960,891 |
| tilisolol | U.S. Pat. No. 4,129,565 |
| timolol | U.S. Pat. No. 3,655,663 |
| toliprolol | U.S. Pat. No. 3,432,545 |
| Xibenolol | U.S. Pat. No. 4,018,824 |

Additional alpha adrenergic blockers which are useful in the combinations of the present invention include, without limitation, those shown in Table 8.

TABLE 8

| Compound Name | Reference |
|---|---|
| amosulalol | U.S. Pat. No. 4,217,307 |
| arotinolol | U.S. Pat. No. 3,932,400 |
| dapiprazole | U.S. Pat. No. 4,252,721 |
| doxazosin | U.S. Pat. No. 4,188,390 |
| fenspirlde | U.S. Pat. No. 3,399,192 |
| indoramin | U.S. Pat. No. 3,527,761 |
| labetalol | U.S. Pat. No. 4,012,444 |
| naftopidil | U.S. Pat. No. 3,997,666 |
| nicergoline | U.S. Pat. No. 3,228,943 |
| prazosin | U.S. Pat. No. 3,511,836 |
| tamsulosin | U.S. Pat. No. 4,703,063 |
| Tolazoline | U.S. Pat. No. 2,161,938 |
| Trimazosin | U.S. Pat. No. 3,669,968 |
| Yohimbine | Raymond-Hamet, J. Pharm. Chim., |
| | 19, 209 (1934) |

Additional angiotensin II receptor antagonists, which are useful in the combinations of the present invention include, without limitation, those shown in Table 9.

TABLE 9

| Compound Name | Reference |
|---|---|
| Candesartan | U.S. Pat. No. 5,196,444 |
| Eprosartan | U.S. Pat. No. 5,185,351 |
| Irbesartan | U.S. Pat. No. 5,270,317 |
| Losartan | U.S. Pat. No. 5,138,069 |
| Valsartan | U.S. Pat. No. 5,399,578 |

Additional vasodilators which are useful in the combinations of the present invention include, without limitation, those shown in Table 10.

TABLE 10

| Compound Name | Reference |
|---|---|
| aluminum nicotinate | U.S. Pat. No. 2,970,082 |
| amotriphene | U.S. Pat. No. 3,010,965 |
| bamethan | Corrigan et al., Journal of the |
| | American Chemical Society, 1945, |
| | 67, 1894 |
| bencyclane | Hungarian Patent No. 151,865 |
| bendazol | J. Chem. Soc., 1968, 2426 |
| benfurodil hemisuccinate | U.S. Pat. No. 3,355,463 |
| benziodarone | U.S. Pat. No. 3,012,042 |
| betahistine | Walter et al., Journal of the American |
| | Chemical Society, 1941, 63, 2771 |
| bradykinin | Hamburg et al., Arch. Biochem. |
| | Biophys., 1958, 76, 252 |
| brovincamine | U.S. Pat. No. 4,146,643 |
| bufeniode | U.S. Pat. No. 3,542,870 |
| buflomedil | U.S. Pat. No. 3,895,030 |
| butalamine | U.S. Pat. No. 3,338,899 |
| cetiedil | French Patent No. 1,460,571 |
| chloracizine | British Patent No. 740,932 |
| chromonar | U.S. Pat. No. 3,282,938 |
| ciclonicate | German Patent No. 1,910,481 |
| cinepazide | Belgian Patent No. 730,345 |
| cinnarizine | U.S. Pat. No. 2,882,271 |
| citicoline | Kennedy et al., Journal of the |
| | American Chemical Society, 1955, |
| | 77, 250 or synthesized as disclosed in |
| | Kennedy, Journal of Biological |
| | Chemistry, 1956, 222, 185 |
| clobenfural | British Patent No. 1,160,925 |
| clonitrate | see Annalen, 1870, 155, 165 |
| cloricromen | U.S. Pat. No. 4,452,811 |
| cyclandelate | U.S. Pat. No. 2,707,193 |
| diisopropylamine | Neutralization of dichloroacetic acid |
| dichloroacetate | with diisopropyl amine |
| diisopropylamine | British Patent No. 862,248 |
| dichloroacetate | |
| dilazep | U.S. Pat. No. 3,532,685 |
| dipyridamole | British Patent No. 807,826 |
| droprenilamine | German Patent No. 2,521,113 |
| ebumamonine | Hermann et at., Journal of the |
| | American Chemical Society, 1979, |
| | 101, 1540 |
| efloxate | British Patent Nos. 803,372 and |
| | 824,547 |
| eledoisin | British Patent No. 984,810 |
| erythrityl | May be prepared by nitration of |
| | erythritol according to methods well- |
| | known to those skilled in the art. See |
| | e.g., Merck Index. |
| etafenone | German Patent No. 1,265,758 |
| fasudil | U.S. Pat. No. 4,678,783 |
| fendiline | U.S. Pat. No. 3,262,977 |
| fenoxedil | U.S. Pat. No. 3,818,021 or German |
| | Patent No. 1,964,712 |
| floredil | German Patent No. 2,020,464 |
| flunarizine | German Patent No. 1,929,330 or |
| | French Patent No. 2,014,487 |
| flunarizine | U.S. Pat. No. 3,773,939 |
| ganglefene | U.S.S.R. Patent No. 115,905 |
| hepronicate | U.S. Pat. No. 3,384,642 |
| hexestrol | U.S. Pat. No. 2,357,985 |
| hexobendine | U.S. Pat. No. 3,267,103 |
| ibudilast | U.S. Pat. No. 3,850,941 |
| ifenprodil | U.S. Pat. No. 3,509,164 |
| iloprost | U.S. Pat. No. 4,692,464 |
| inositol | Badgett et al., Journal of the |
| | American Chemical Society, 1947, |
| | 69, 2907 |
| isoxsuprine | U.S. Pat. No. 3,056,836 |
| itramin tosylate | Swedish Patent No. 168,308 |
| kallidin | Biochem. Biophys. Re&Commun., |
| | 1961, 6, 210 |
| kallikrein | German Patent No. 1,102,973 |
| khellin | Baxter et al., Journal of the Chemical |
| | Society, 1949, S 30 |
| lidofiazine | U.S. Pat. No. 3,267,104 |
| lomerizine | U.S. Pat. No. 4,663,325 |
| mannitol hexanitrate | May be prepared by the nitration of |
| | mannitol according to methods well- |
| | known to those skilled in the art |
| medibazine | U.S. Pat. No. 3,119,826 |
| moxisylyte | German Patent No. 905,738 |
| nafronyl | U.S. Pat. No. 3,334,096 |
| nicametate | Blicke & Jenner, J. Am. Chem. Soc., |
| | 64, 1722 (1942) |
| nicergoline | U.S. Pat. No. 3,228,943 |
| nicofuranose | Swiss Patent No. 366,523 |
| nimodipine | U.S. Pat. No. 3,799,934 |
| nitroglycerin | Sobrero, Ann., 64, 398 (1847) |
| nylidrin | U.S. Pat. Nos. 2,661,372 and |
| | 2,661,373 |
| papaverine | Goldberg, Chem. Prod. Chem. News, |
| | 1954, 17, 371 |
| pentaerythritol tetranitrate | U.S. Pat. No. 2,370,437 |
| pentifylline | German Patent No. 860,217 |
| pentoxifylline | U.S. Pat. No. 3,422,107 |
| pentrinitrol | German Patent No. 638,422-3 |
| perhexilline | British Patent No. 1,025,578 |
| pimefylline | U.S. Pat. No. 3,350,400 |
| piribedil | U.S. Pat. No. 3,299,067 |
| prenylamine | U.S. Pat. No. 3,152,173 |
| propatyl nitrate | French Patent No. 1,103,113 |
| prostaglandin El | May be prepared by any of the |
| | methods referenced in the Merck |
| | Index, Twelfth Edition, Budaved, |
| | Ed., New Jersey, 1996, p. 1353 |
| suloctidil | German Patent No. 2,334,404 |
| tinofedrine | U.S. Pat. No. 3,563,997 |
| tolazoline | U.S. Pat. No. 2,161,938 |
| trapidil | East German Patent No. 55,956 |
| tricromyl | U.S. Pat. No. 2,769,015 |
| trimetazidine | U.S. Pat. No. 3,262,852 |
| trolnitrate phosphate | French Patent No. 984,523 or |
| | German Patent No. 830,955 |
| vincamine | U.S. Pat. No. 3,770,724 |
| vinpocetine | U.S. Pat. No. 4,035,750 |
| Viquidil | U.S. Pat. No. 2,500,444 |
| Visnadine | U.S. Pat. Nos. 2,816,118 and |
| | 2,980,699 |
| xanthinol niacinate | German Patent No. 1,102,750 or |
| | Korbonits et al., Acta. Pharm. Hung., |
| | 1968, 38, 98 |

Additional diuretics which are useful in the combinations of the present invention include, without limitation, those shown in Table 11.

TABLE 11

| Compound Name | Reference |
|---|---|
| Acetazolamide | U.S. Pat. No. 2,980,676 |
| Althiazide | British Patent No. 902,658 |
| Amanozine | Austrian Patent No. 168,063 |
| Ambuside | U.S. Pat. No. 3,188,329 |
| Amiloride | Belgian Patent No. 639,386 |
| Arbutin | Tschb&habln, Annalen, 1930, 479, |
| | 303 |
| Azosemide | U.S. Pat. No. 3,665,002 |
| Bendroflumethiazide | U.S. Pat. No. 3,265,573 |
| Benzthiazide | McManus et al., 136th Am. Soc. |
| | Meeting (Atlantic City, September |
| | 1959). Abstract of Papers, pp 13-0 |
| benzylhydro-chlorothiazide | U.S. Pat. No. 3,108,097 |
| Bumetanide | U.S. Pat. No. 3,634,583 |
| Butazolamide | British Patent No. 769,757 |
| Buthiazide | British Patent Nos. 861,367 and |
| | 885,078 |
| Chloraminophenamide | U.S. Pat. Nos. 2,809,194, |
| | 2,965,655 and 2,965,656 |
| Chlorazanil | Austrian Patent No. 168,063 |
| Chlorothiazide | U.S. Pat. Nos. 2,809,194 and |
| | 2,937,169 |
| Chlorthalidone | U.S. Pat. No. 3,055,904 |
| Clofenamide | Olivier, Rec. Trav. Chim., 1918, 37, |
| | 307 |
| Clopamide | U.S. Pat. No. 3,459,756 |
| Clorexolone | U.S. Pat. No. 3,183,243 |
| Cyclopenthiazide | Belgian Patent No. 587,225 |
| Cyclothiazide | Whitehead et al., Journal of Organic |
| | Chemistry, 1961, 26, 2814 |
| Disulfamide | British Patent No. 851,287 |
| Epithiazide | U.S. Pat. No. 3,009,911 |
| ethacrynic acid | U.S. Pat. No. 3,255,241 |
| Ethiazide | British Patent No. 861,367 |
| Ethoxolamide | British Patent No. 795,174 |
| Etozolin | U.S. Pat. No. 3,072,653 |
| Fenquizone | U.S. Pat. No. 3,870,720 |
| Furosemide | U.S. Pat. No. 3,058,882 |
| Hydracarbazine | British Patent No. 856,409 |
| Hydrochlorothiazide | U.S. Pat. No. 3,164,588 |
| Hydroflumethiazide | U.S. Pat. No. 3,254,076 |
| Indapamide | U.S. Pat. No. 3,565,911 |
| Isosorbide | U.S. Pat. No. 3,160,641 |
| Mannitol | U.S. Pat. No. 2,642,462; or |
| | 2,749,371; or 2,759,024 |
| Mefruside | U.S. Pat. No. 3,356,692 |
| Methazolamide | U.S. Pat. No. 2,783,241 |
| Methyclothiazide | Close et al., Journal of the American |
| | Chemical Society, 1960, 82, 1132 |
| Meticrane | French Patent Nos. M2790 and |
| | 1,365,504 |
| Metochalcone | Freudenberg et al., Ber., 1957, 90, |
| | 957 |
| Metolazone | U.S. Pat. No. 3,360,518 |
| Muzolimine | U.S. Pat. No. 4,018,890 |
| Paraflutizide | Belgian Patent No. 620,829 |
| Perhexiline | British Patent No. 1,025,578 |
| Piretanide | U.S. Pat. No. 4,010,273 |
| Polythiazide | U.S. Pat. No. 3,009,911 |
| Quinethazone | U.S. Pat. No. 2,976,289 |
| Teclothiazide | Close et al., Journal of the American |
| | Chemical Society, 1960, 82, 1132 |
| Ticrynafen | U.S. Pat. No. 3,758,506 |
| Torasemide | U.S. Pat. No. 4,018,929 |
| Triamterene | U.S. Pat. No. 3,081,230 |
| Trichlormethiazide | deStevens et al., Experientia, 1960, |
| | 16, 113 |
| Tripamide | Japanese Patent No. 73 05,585 |
| Urea | Can be purchased from commercial |
| | sources |
| Xipamide | U.S. Pat. No. 3,567,777 |

### EXAMPLE 1

Compound A is a lipophilic vascular protectant with strong antioxidant properties equipotent to probucol, but without the undesired QT prolongation side effect. A study was conducted to determine whether Compound A reduces restenosis as assessed by intravascular ultrasound (IVUS) when administered for 2 weeks before and 4 weeks after percutaneous coronary intervention (PCI) with or without stent placement. A multi-center, double-blind placebo-controlled randomized trail with 5 treatment groups:

1. Placebo;

2. 500 mg of probucol, twice daily;

3. 70 mg of Compound A, once daily;

4. 140 mg of Compound A, once daily;

5. 280 mg of Compound A, once daily.

Fifty variables were evaluated, including diabetes, hypertension, smoking, angina class, prior MI, CABG, PCI, and the number of diseased vessels per patient to determine baseline characteristics. No baseline differences between the five study groups, including the distribution of target vessels, which was similar among all groups.

Treatment of the above was given 2 weeks prior to and 4 weeks after PCI. PCI was performed on greater than or equal to 1 native artery with greater than or equal to 1 de novo lesion of greater than or equal to 50%. All PCI procedures (with or without stent placement) and post-PCI management were performed according to recognized current clinical practice. 0.3 mg of nitroglycerin(Ntg) intracoronary (IC) were administered at every angiogram. Quantitative Coronary Angiography (QCA) measurements were taken prior to-PCI, 10 minutes post-PCI, and final follow-up (approximately 6 months).

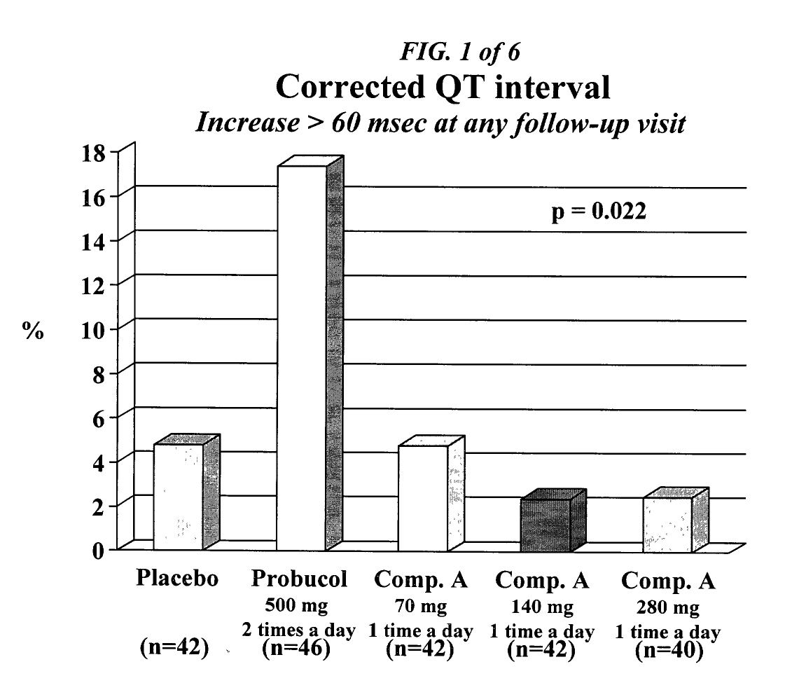

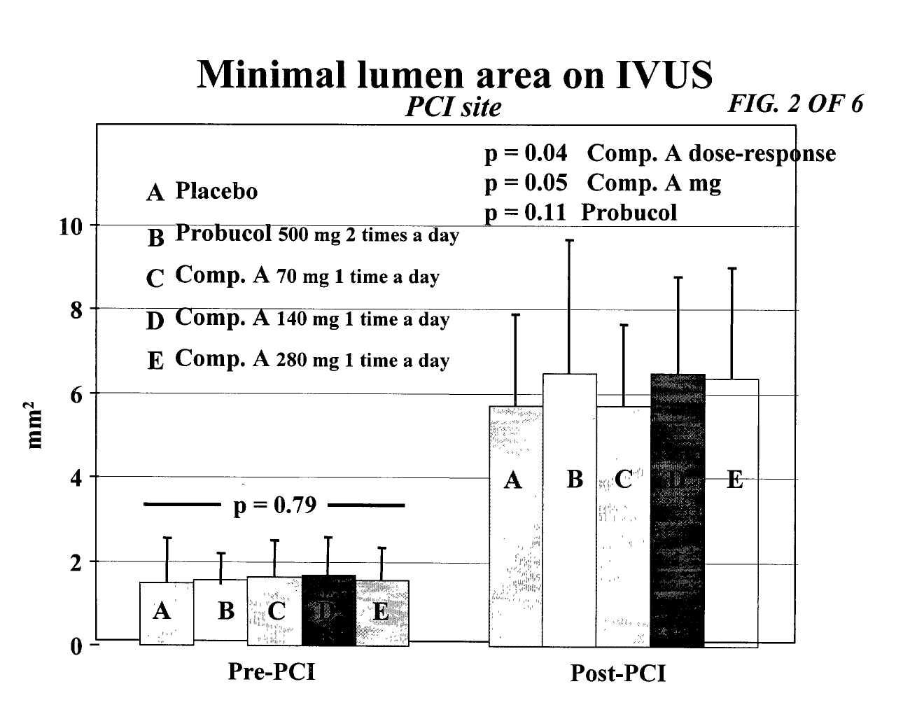

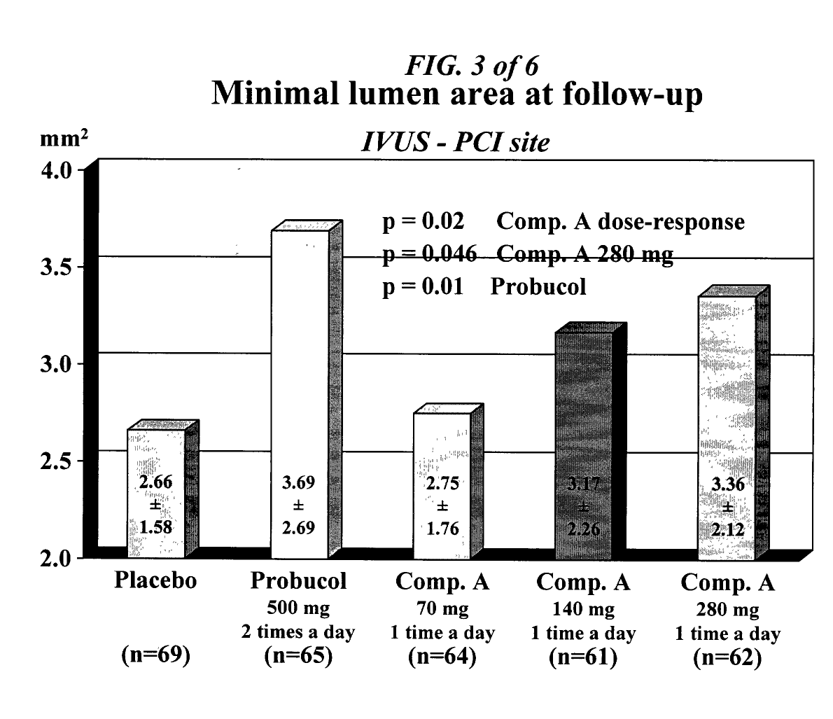

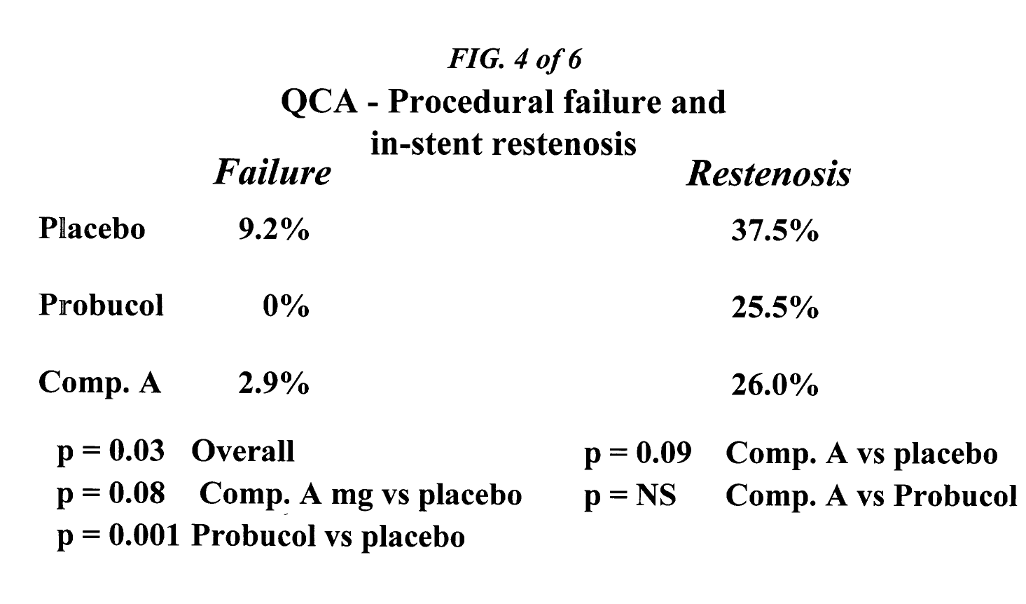

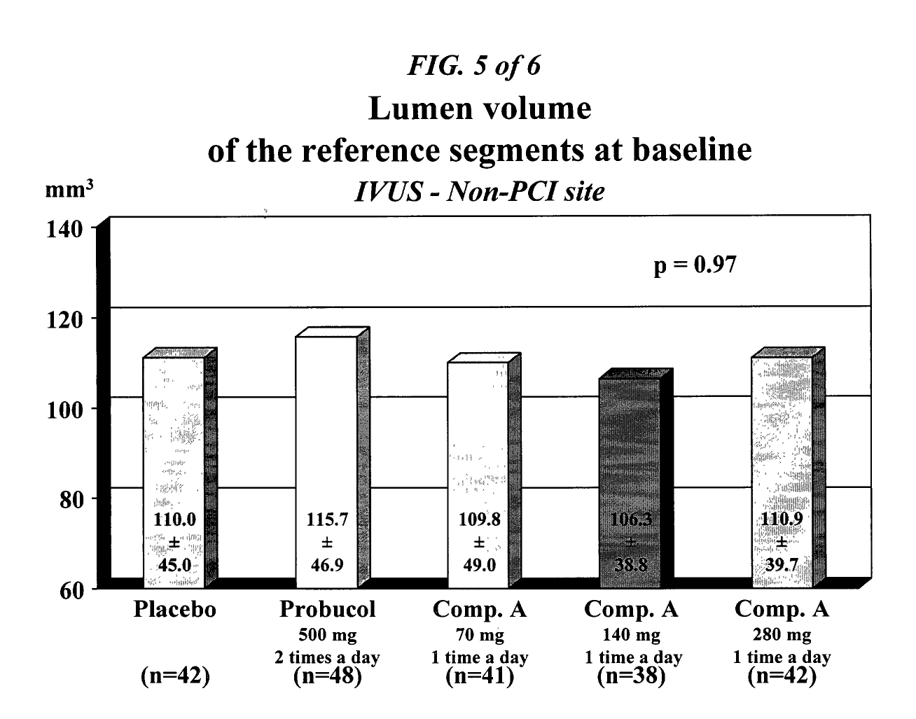

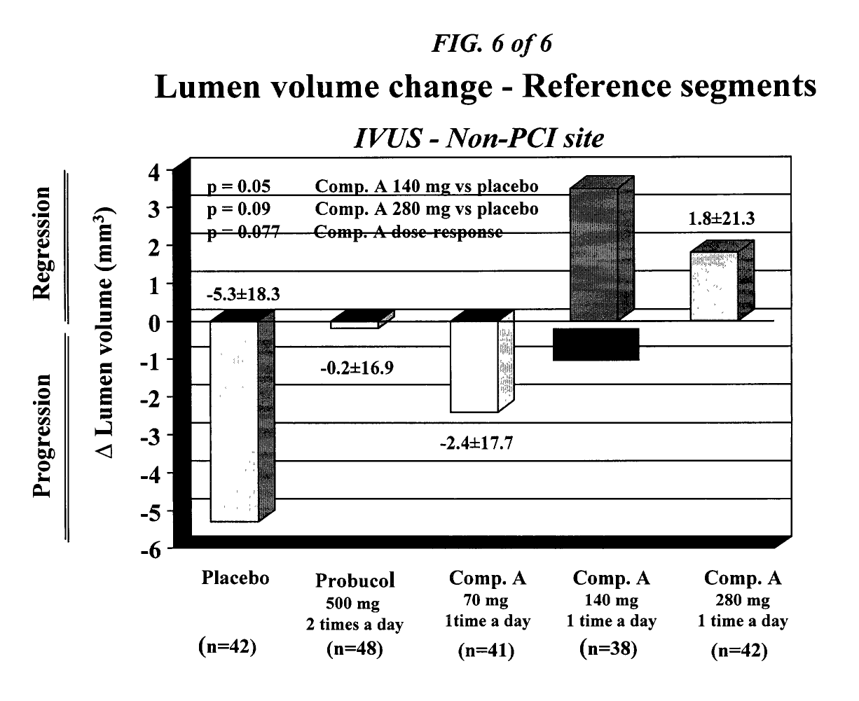

IVUS examinations were conducted at 30 Mhz using 3.5 French CVIS catheters. All IVUS examinations were preceded by IC Ntg 0.3 mg. Results of these examinations are found in FIGS. 1-6.

Compound A and probucol were shown to reduce restenosis after PCI. In contrast to probucol, Compound A resulted in the surprising improvement of lumen dimensions of the reference segments, without causing prolongation of the QTc interval. Clearly, prolonged therapy with Compound A would result in the prevention of restenosis, but more importantly, treatment with Compound A would reverse or prevent diseases of the cardiovascular characterized by a decreased lumen diameter including atherosclerosis.

### EXAMPLE 2

Table A shows a comparison of preclinical effects of the compound A with probucol. Although their antioxidant activities are essentially equal, compound A is a very active inhibitor of VCAM-1 and MCP-1 gene expression, even at low micromolar concentrations, whereas probucol was shown to be inactive even at very high concentrations. As an anti-inflammatory agent, compound A is consistently very effective while the activity of probucol is highly variable.

TABLE A

| Activities | Compound A | Probucol |
|---|---|---|
| Antioxidant | +++ | +++ |
| VCAM-1 Expression Inhibitor | +++ | − |
| Anti-Inflammatory | +++ | +/− |
| LDL-Lowering | +++ | +/− |
| HDL-Lowering | +/− | +++ |
| Anti-atherosclerotic Effects: | | |
| Rabbits | +++ | + |
| LDLr-KO mice | +++ | + |
| ApoE-KO mice | +++ | + |
| QTc prolongation | − | +++ |
| Inhibition of Xanthoma Progression | +++ | + |

Synthesis of the Compounds

The compounds employed in the present invention can be manufactured by those skilled in the art by using the procedures set forth in U.S. Pat. Nos. 6,147,250 and 6,323,359. In particular, Compound A, Butanedioic acid, mono [4-[[1-[[3,5-bis(1,1-dimethylethyl)-4-hydroxyphenyl]thio]-1-methylethyl]thio]2,6-bis(1,1-dimethylethyl)phenyl]ester, can be made using the following procedure:

To an appropriately sized, nitrogen-purged, glass reaction vessel is charged 375 mL anhydrous (0.01% water) tetrahydrofuran (THF) at 20-25° C. To the stirred THF solution is added 23.44 g, 199 mmol, 2.14 equivalents of potassium butoxide (KOtBu). To the resulting hazy solution is added 48.5 g, 93 mmol, 1.0 equivalent of 99% pure probucol in three equal portions. The orange-yellow colored solution is stirred for 45 minutes. A temperature drop from about 35° to about 22° C. is noted. To this solution is added 32.9 g, 326 mmol, 3.5 equivalents of succinic anhydride (SSA) over a period of about 90 seconds. The solution color first becomes brown and then deep blue. A temperature of about 25° results. Analysis by HPLC of the reaction mixture at this point shows a ratio of 3:10:7 disuccinyl probucol (DSP): monosuccinyl probucol (MSP): probucol (PRO). After washing twice with 12-14% sodium hydroxide, the solution is concentrated to about 25% of its original volume under reduced pressure at 45° C. The resulting slurry is diluted with 110 mL heptanes, and concentrated under reduced pressure two times. The final slurry amounts to about 150 mL of material. It is diluted with 400 mL heptanes, cooled to 0-5° C. with stiring and vacuum filtered. The residue is washed with 250 mL heptanes and, to the wet cake is added 65 mL tert-butylmethyl ether (MBTE) with string. The resulting slurry is filtered, the residue washed with 23 mL MBTE and the filtrate washed with 40 mL 1.3 N hydrochloric acid containing 2.5 g sodium chloride. The solution is dried azeotropically at 40° C. with the addition of about 200 mL of MBTE. The resulting residue is diluted with 200 mL heptanes, warmed to 70° C. and seeded with 15 mg MSP. After cooling the seeded solution to 5° C. over a period of about 18 hours, the cold slurry is filtered, washed with 100 mL heptanes and dried to yield off-white solid MSP, 23.2 g, 40.1 mmol %, 98.7 AP. The filtrate, containing free probucol is treated by concentrating to about 350 mL, washed with 40 mL 1 N HCl and further concentrated to about 80 mL at 75° C. under reduced pressure. The solution is seeded and cooled to about 0-5° C. and held at this temperature overnight. Filtration, washing the residue with heptanes and drying produces white, crystalline probucol, 10.33 g, 21.3 mol %, 99.91 AP. The mother liquor provides an additional 6.1 g, 12.6 mol %, 99.91 AP of probucol.

While in the foregoing specification this invention has been described related to many embodiments, it will be apparent to those skilled in the art that the invention is susceptible to additional embodiments and that certain of the details described herein may be varied considerably without departing from the basic principles of the invention.

We claim:

1. A method to prevent cardiovascular disease characterized by a decrease in lumen diameter comprising administering a therapeutically effective amount of a compound having the formula 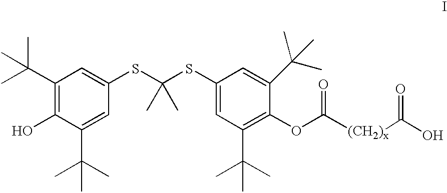
wherein x is selected from 1, 2, 3 or 4;
or a pharmaceutically acceptable salt, ester or prodrug thereof to a mammal.

2. The method of claim 1, wherein the compound is 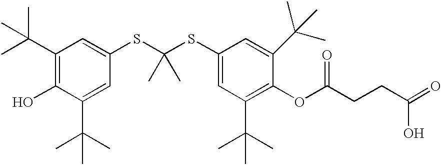
or a pharmaceutically acceptable salt, ester or prodrug thereof.

3. The method of claim 1 or 2, wherein the mammal is human.

4. A method to reverse cardiovascular disease characterized by a decrease in lumen diameter comprising administering a therapeutically effective amount of a compound having the formula 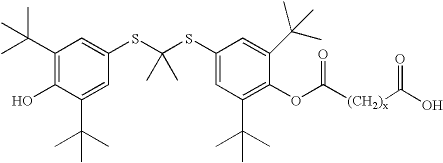
wherein x is selected from 1, 2, 3 or 4;
or a pharmaceutically acceptable salt, ester or prodrug thereof, to a mammal.

5. The method of claim 4, wherein the compound is 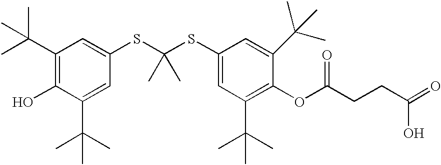
or a pharmaceutically acceptable salt, ester or prodrug thereof.

6. The method of claim 4 or 5, wherein the mammal is a human.

7. A method to prevent progression of a cardiovascular disease characterized by a decrease in lumen diameter comprising administering a therapeutically effective amount of a compound to a mammal, wherein the compound having the formula 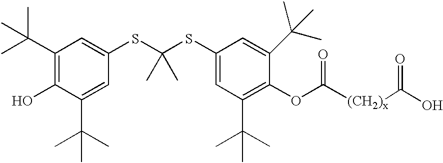
wherein x is selected from 1, 2, 3 or 4;
or a pharmaceutically acceptable salt, ester or prodrug thereof.

8. The method of claim 7 wherein the compound is 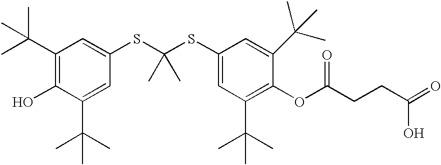
or a pharmaceutically acceptable salt, ester or prodrug thereof.

9. The method of claim 7 or 8, wherein the mammal is a human.

10. A method to prevent cardiovascular disease characterized by a decrease in lumen diameter comprising administering a therapeutically effective amount of a compound having the formula 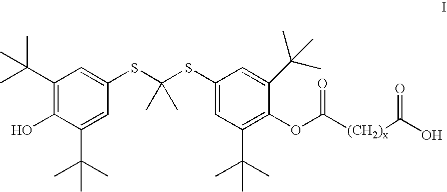
wherein x is selected from 1, 2, 3 or 4;
or a pharmaceutically acceptable salt, ester or prodrug thereof, in alternation or combination with other therapeutic agents providing a complimentary effect to a mammal.

11. The method of claim 10, wherein the compound is 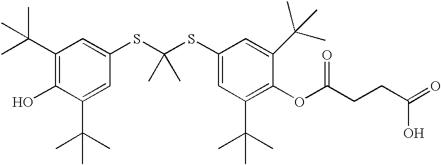
or a pharmaceutically acceptable salt, ester or prodrug thereof.

12. The method of claim 10 or 11, wherein the mammal is human.

13. A method to reverse cardiovascular disease characterized by a decrease in lumen diameter comprising administering a therapeutically effective amount of a compound having the formula 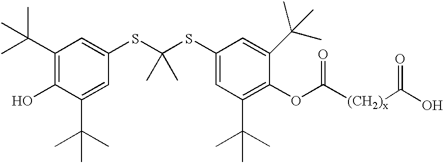
wherein x is selected from 1, 2, 3 or 4;
or a pharmaceutically acceptable salt, ester or prodrug thereof, in alternation or in combination with other therapeutic agents providing a complimentary effect to a mammal.

14. The method of claim 13, wherein the compound is 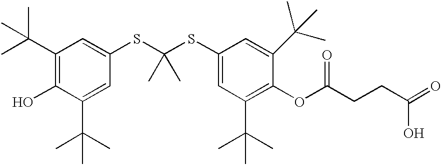
or a pharmaceutically acceptable salt, ester or prodrug thereof.

15. The method of claim 13 or 14, wherein the mammal is a human.

16. A method to prevent progression of a cardiovascular disease characterized by a decrease in lumen diameter comprising administering a therapeutically effective amount of a compound to a mammal, wherein the compound having the formula 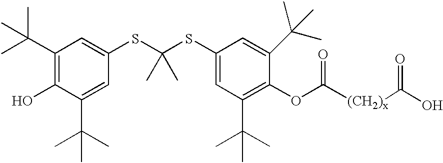
wherein x is selected from 1, 2, 3 or 4;
or a pharmaceutically acceptable salt, ester or prodrug thereof, in alternation or combination with other therapeutic agents providing a complimentary effect.

17. The method of claim 16, wherein the compound is 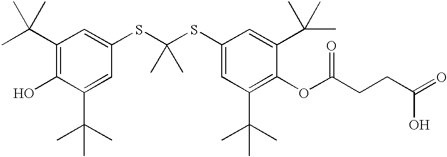
or a pharmaceutically acceptable salt, ester or prodrug thereof.

18. The method of claim 16 or 17, wherein the mammal is a human.

19. The method as in any one of claims 10-18, wherein the therapeutic agent is probucol.

20. The method as in any one of claims 10-18, wherein the therapeutic agent is statin.

21. The method as in any one of claims 10-18, wherein the therapeutic agent is an IBAT inhibitor.

22. A method to increase the lumen diameter of a blood vessel in a human, comprising administering an effective lumen diameter increasing amount of a compound having the formula: 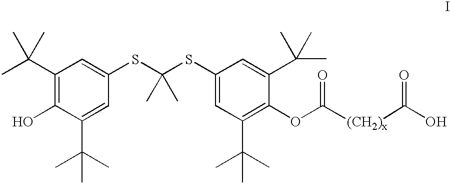
wherein x is selected from 1, 2, 3 or 4;
or a pharmaceutically acceptable salt, ester or prodrug thereof to a human.
